SEQUENCE LISTING (12 sequences)

SEQ ID NO: 1 - protein, 641 aa, Homo Sapien
MAKAAAIGIDLGTTYSCVGVFQHGKVEIIANDQGNRTTPSYVAFTDTERLIGDAAKNQVALNPQNTVFDAKRLIGRKFGDPVVQSDMKHWPFQVINDGDKPKVQVSYKGDTKAFYPEEISSMVLTKMKEIAEAYLGYPVTNAVITVPAYFNDSQRQATKDAGVIAGLNVLRIINEPTAAAIAYGLDRTGKGERNVLIFDLGGGTFDVSILTIDDGIFEVKATAGDTHLGGEDFDNRLVNHFVEEFKRKHKKDISQNKRAVRRLRTACERAKRTLSSSTQASLEIDSLFEGIDFYTSITRARFEELCSDLFRSTLEPVEKALRDAKLDKAQIHDLVLVGGSTRIPKVQKLLQDFFNGRDLNKSINPDEAVAYGAAVQAAILMGDKSENVQDLLLLDVAPLSLGLETAGGVMTALIKRNSTIPTKQTQIFTTYSDNQPGVLIQVYEGERAMTKDNNLLGRFELSGIPPAPRGVPQIEVTFDIDANGILNVTATDKSTGKANKITITNDKGRLSKEEIERMVQEAEKYKAEDEVQRERVSAKNALESYAFNMKSAVEDEGLKGKISEADKKKVLDKCQEVISWLDANTLAEKDEFEHKRKELEQVCNPIISGLYQGAGGPGPGGFGAQGPKGGSGSGPTIEEVD

SEQ ID NO: 2 - protein, 641 aa, Homo Sapien
MAKAAAIGIDLGTTYSCVGVFQHGKVEIIANDQGNRTTPSYVAFTDTERLIGDAAKNQVALNPQNTVFDAKRLIGRKFGDPVVQSDMKHWPFQVINDGDKPKVQVSYKGETKAFYPEEISSMVLTKMKEIAEAYLGYPVTNAVITVPAYFNDSQRQATKDAGVIAGLNVLRIINEPTAAAIAYGLDRTGKGERNVLIFDLGGGTFDVSILTIDDGIFEVKATAGDTHLGGEDFDNRLVNHFVEEFKRKHKKDISQNKRAVRRLRTACERAKRTLSSSTQASLEIDSLFEGIDFYTSITRARFEELCSDLFRSTLEPVEKALRDAKLDKAQIHDLVLVGGSTRIPKVQKLLQDFFNGRDLNKSINPDEAVAYGAAVQAAILMGDKSENVQDLLLLDVAPLSLGLETAGGVMTALIKRNSTIPTKQTQIFTTYSDNQPGVLIQVYEGERAMTKDNNLLGRFELSGIPPAPRGVPQIEVTFDIDANGILNVTATDKSTGKASKITITNDKGRLSKEEIERMVQEAEKYKAEDEVQRERVSAKNALESYAFNMKSAVEDEGLKGKISEADKKKVLDKCQEVISWLDANTLAEKDEFEHKRKELEQVCNPIISGLYQGAGGPGPGGFGAQGPKGGSGSGPTIEEVD

SEQ ID NO: 3 - protein, 638 aa, Cercopithecus Aethiops
MAKAAAIGIDLGTTYSCVGVFQHGKVEIIANDQGNRTTPSYVAFTDTERLIGDAAKNQVALNPQNTVFDAKRLIGRKFGDPVVQSDMKHWPFQVINDGDKPKVQVSYKGETKAFYPEEISSMVLTKMKEIAEADLGYPVTNAVITVPAYFNDSQRQATKDAGVIAGLNVLRIINEPTRTIAYALDRTGKGERNVLIFDLGGGTFDVSILTIDDGIFEVKATAGDTTWVEDFDNRLVNHFVEEFKRKHKKDISQNKRAVRRLRTACERAKRTLSSSTQASLEIDSLFEGIDFYTSITRARFEELCSDLFRSTLEPVEKALRDAKLDKAQIHDLVLVGGSTRIPKVQKLLQDFFNGRDLNKSINPDEAVAYGAAVQAAILMGDKSENVQDLLLLDVAPLSLGLETPGGVMTALIKRNSTIPTKQTQIFTTYSDNQPGVLIQVYEGERAMTKDNNLLGRFELSGIPPAPGVPQIEVTFEIDANGILNVTATDKSTGKANKITITNDKGRLSKEEIERMVQEAEKYKAEDEVQRERVSAKNALESYALNMKSAVEDEGLKGKISEADKKKVLDKCQEVISWLDANTLAEKDEFEHKRKELEQVCNPIISGLYQGGGGPGPGGFGAQGPKGGSGSGPTIEEVD

SEQ ID NO: 4 - protein, 642 aa, Mus Musculus
MAKNTAIGIDLGTTYSCVGVFQHGKVEIIANDQGNRTTPSYVAFTDTERLIGDAAKNQVALNPQNTVFDAKRLIGRKFGDAVVQSDMKHWPFQVVNDGDKPKVQVNYKGESRSFFPEEISSMVLTKMKEIAEAYLGHPVTNAVITVPAYFNDSQRQATKDAGVIAGLNVLRIINEPTAAAIAYGLDRTGKGERNVLIFDLGGGTFDVSILTIDDGIFEVKATAGDTHLGGEDFDNRLVSHFVEEFKRKHKKDISQNKRAVRRLRTACERAKRTLSSSTQASLEIDSLFEGIDFYTSITRARFEELCSDLFRGTLEPVEKALRDAKMDKAQIHDLVLVGGSTRIPKVQKLLQDFFNGRDLNKSINPDEAVAYGAAVQAAILMGDKSENVQDLLLLDVAPLSLGLETAGGVMTALIKRNSTIPTKQTQTFTTYSDNQPGVLIQVYEGERAMTRDNNLLGRFELSGIPPAPRGVPQIEVTFDIDANGILNVTATDKSTGKANKITITNDKGRLSKEEIERMVQEAERYKAEDEVQRDRVAAKNALESYAFNMKSAVEDEGLKGKLSEADKKKVLDKCQEVISWLDSNTLADKEEFVHKREELERVCSPIISGLYQGAGAPGAGGFGAQAPPKGASGSGPTIEEVD

SEQ ID NO: 5 - protein, 641 aa, Mus Musculus
MAKNTAIGIDLGTTYSCVGVFQHGKVEIIANDQGNRTTPSYVAFTDTERLIGDAAKNQVALNPQNTVFDAKRLIGRKFGDAVVQSDMKHWPFQVVNDGDKPKVQVNYKGESRSFFPEEISSMVLTKMKEIAEAYLGHPVTNAVITVPAYFNDSQRQATKDAGVIAGLNVLRIINEPTAAAIAYGLDRTGKGERNVLIFDLGGGTFDVSILTIDDGIFEVKATAGDTHLGGEDFDNRLVSHFVEEFKRKHKKDISQNKRAVRRLRTACERAKRTLSSSTQASLEIDSLFEGIDFYTSITRARFEELCSDLFRGTLEPVEKALRDAKMDKAQIHDLVLVGGSTRIPKVQKLLQDFFNGRDLNKSINPDEAVAYGAAVQAAILMGDKSENVQDLLLLDVAPLSLGLETAGGVMTALIKRNSTIPTKQTQTFTTYSDNQPGVLIQVYEGERAMTRDNNLLGRFELSGIPPAPRGVPQIEVTFDIDANGILNVTATDKTTGKANKITITNDKGRLSKEEIERMVQEAERYKAEDEVQRDRVAAKNALESYAFNMKSAVEDEGLKGKLSEADKKKVLDKCQEVISWLDSNTLADKEEFVHKREELERVCSPIISGLYQGAGAPGAGGFGAQAPKGASGSGPTIEEVD

SEQ ID NO: 6 - protein, 641 aa, Rattus Norvegicus
MAKKTAIGIDLGTTYSCVGVFQHGKVEIIANDQGNRTTPSYVAFTDTERLIGDAAKNQVALNPQNTVFDAKRLIGRKFGDPVVQSDMKHWPFQVVNDGDKPKVQVNYKGENRSFYPEEISSMVLTKMKEIAEAYLGHPVTNAVITVPAYFNDSQRQATKDAGVIAGLNVLRIINEPTAAAIAYGLDRTGKGERNVLIFDLGGGTFDVSILTIDDGIFEVKATAGDTDLGGEDFDNRLVSHFVEEFKRKHKKDISQNKRAVRRLRTACERAKRTLSSSTQASLEIDSLFEGIDFYTSITRARFEELCSDLFRGTLEPVEKALRDAKLDKAQIHDLVLVGGSTRIPKVQKLLQDFFNGRDLNKSINPDEAVAYGAAVQAAILMGDKSENVQDLLLLDVAPLSLGLETAGGVMTALIKRNSTIPTKQTQTFTTYSDNQPGVLIQVYEGERAMTRDNNLLGRFELSGIPPAPRGVPQIEVTFDIDANGILNVTATDKSTGKANKITITNDKGRLSKEEIERMVQEAERYKAEDEVQRERVAAKNALESYAFNMKSAVEDEGLKGKISEADKKKVLDKCQEVISWLDSNTLAEKEEFVHKREELERVCNPIISGLYQGAGAPGAGGFGAQAPKGGSGSGPTIEEVD

SEQ ID NO: 7 - protein, 641 aa, Rattus Norvegicus
MAKKTAIGIDLGTTYSCVGVFQHGKVEIIANDQGNRTTPSYVAFTDTERLIGDAAKNQVALNPQNTVFDAKRLIGRKFGDPVVQSDMKHWPFQVVNDGDKPKVQVNYKGENRSFYPEEISSMVLTKMKEIAEAYLGHPVTNAVITVPAYFNDSQRQATKDAGVIAGLNVLRIINEPTAAAIAYGLDRTGKGERNVLIFDLGGGTFDVSILTIDDGIFEVKATAGDTHLGGEDFDNRLVSHFVEEFKRKHKKDISQNKRAVRRLRTACERAKRTLSSSTQASLEIDSLFEGIDFYTSITRARFEELCSDLFRGTLEPVEKALRDAKLDKAQIHDLVLVGGSTRIPKVQKLLQDFFNGRDLNKSINPDEAVAYGAAVQAAILMGDKSENVQDLLLLDVAPLSLGLETAGGVMTALIKRNSTIPTKQTQTFTTYSDNQPGVLIQVYEGERAMTRDNNLLGRFELSGIPPAPRGVPQIEVTFDIDANGILNVTATDKSTGKANKITITNDKGRLSKEEIERMVQEAERYKAEDEVQRERVAAKNALESYAFNMKSAVEDEGLKGKISEADKKKVLDKCQEVISWLDSNTLAEKEEFVHKREELERVCNPIISGLYQGAGAPGAGGFGAQAPKGGSGSGPTIEEVD

SEQ ID NO: 8 - protein, 641 aa, Bos Taurus
MAKNTAIGIDLGTTYSCVGVFQHGKVEIIANDQGNRTTPSYVAFTDTERLIGDAAKNQVALNPQNTVFDAKRLIGRKFGDPVVQSDMKHWPFRVINDGDKPKVQVSYKGETKAFYPEEISSMVLTKMKEIAEAYLGHPVTNAVITVPAYFNDSQRQATKDAGVIAGLNVLRIINEPTAAAIAYGLDRTGKGERNVLIFDLGGGTFDVSILTIDDGIFEVKATAGDTHLGGEDFDNRLVNHFVEEFKRKHKKDISQNKRAVRRLRTACERAKRTLSSSTQASLEIDSLFEGIDFYTSITRARFEELCSDLFRSTLEPVEKALRDAKLDKAQIHDLVLVGGSTRIPKVQKLLQDFFNGRDLNKSINPDEAVAYGAAVQAAILMGDKSENVQDLLLLDVAPLSLGLETAGGVMTALIKRNSTIPTKQTQIFTTYSDNQPGVLIQVYEGERAMTRDNNLLGRFELSGIPPAPRGVPQIEVTFDIDANGILNVTATDKSTGKANKITITNDKGRLSKEEIERMVQEAEKYKAEDEVQRERVSAKNALESYAFNMKSAVEDEGLKGKISEADKKKVLDKCQEVISWLDANTLAEKDEFEHKRKELEQVCNPIISRLYQGAGGPGAGGFGAQGPKGGSGSGPTIEEVD

SEQ ID NO: 9 - protein, 641 aa, Bos Taurus
MAKNMAIGIDLGTTYSCVGVFQHGKVEIIANDQGNRTTPSYVAFTDTERLIGDAAKNQVALNPQNTVFDAKRLIGRKFGDPVVQSDMKEWPFRVINDGDKPKVQVSYKGETKAFYPEEISSMVLTKMKEIAEAYLGHPVTNAVITVPAYFNDSQRQATKDAGVIAGLNVLRIINEPTAAAIAYGLDRTGKGERNVLIFDLGGGTFDVSILTIDDGIFEVKATAGDTHLGGEDFDNRLVNHFVEEFKRKHKKDISQNKRAVRRLRTACERAKRTLSSSTQASLEIDSLFEGIDFYTSITRARFEELCSDLFRSTLEPVEKALRDAKLDKAQIHDLVLVGGSTRIPKVQKLLQDFFNGRDLNKSINPDEAVAYGAAVQAAILMGDKSENVQDLLLLDVAPLSLGLETAGGVMTALIKRNSTIPTKQTQIFTTYSDNQPGVLIQVYEGERAMTRDNNLLGRFELSGIPPAPRGVPQIEVTFDIDANGILNVTATDKSTGKANKITITNDKGRLSKEEIERMVQEAEKYKAEDEVQRERVSAKNALESYAFNMKSAVEDEGLKGKISEADKKKVLDKCQEVISWLDANTLAEKDEFEHKRKELEQVCNPIISRLYQGAGGPGAGGFGAQGPKGGSGSGPTIEEVD

SEQ ID NO: 10 - protein, 641 aa, Homo Sapien
MATAKGIAIGIDLGTTYSCVGVFQHGKVEIIANDQGNRTTPSYVAFTDTERLIGDAAKNQVAMNPQNTVFDAKRLIGRKFNDPVVQADMKLWPFQVINEGGKPKVLVSYKGENKAFYPEEISSMVLTKLKETAEAFLGHPVTNAVITVPAYFNDSQRQATKDAGVIAGLNVLRIINEPTAAAIAYGLDKGGQGERHVLIFDLGGGTFDVSILTIDDGIFEVKATAGDTHLGGEDFDNRLVSHFVEEFKRKHKKDISQNKRAVRRLRTACERAKRTLSSSTQANLEIDSLYEGIDFYTSITRARFEELCADLFRGTLEPVEKALRDAKMDKAKIHDIVLVGGSTRIPKVQRLLQDYFNGRDLNKSINPDEAVAYGAAVQAAILMGDKSEKVQDLLLLDVAPLSLGLETAGGVMTALIKRNSTIPTKQTQIFTTYSDNQPGVLIQVYEGERAMTKDNNLLGRFDLTGIPPAPRGVPQIEVTFDIDANGILNVTAMDKSTGKVNKITITNDKGRLSKEEIERMVLDAEKYKAEDEVQREKIAAKNALESYAFNMKSVVSDEGLKGKISESDKNKILDKCNELLSWLEVNQLAEKDEFDHKRKELEQMCNPIITKLYQGGCTGPACGTGYVPGRPATGPTIEEVD

SEQ ID NO: 11 - protein, 641 aa, Mus Musculus
MAANKGMAIGIDLGTTYSCVGVFQHGKVEIIANDQGNRTTPSYVAFTDTERLIGDAAKNQVAMNPQNTVFDAKRLIGRKFNDPVVQSDMKLWPFQVINEAGKPKVMVSYKGEKKAFYPEEISSMVLTKMKETAEAFLGHNVTNAVITVPAYFNDSQRQATKDAGVIAGLNVLRIINEPTAAAIAYGLDKGSHGERHVLIFDLGGGTFDVSILTIDDGIFEVKATAGDTHLGGEDFDNRLVSHFVEEFKRKHKKDISQNKRAVRRLRTACERAKRTLSSSTQANLEIDSLYEGIDFYTSITRARFEELCADLFRGTLEPVEKSLRDAKMDKAKIHDIVLVGGSTRIPKVQKLLQDYFNGRDLNKSINPDEAVAYGAAVQAAILMGDKSEKVQDLLLLDVAPLSLGLETAGGVMTVLIKRNSTIPTKQTQIFTTYSDNQPGVLIQVYEGERAMTRDNNLLGRFDLTGIPPAPRGVPQIEVTFDIDANGILNVTAMDKSTGKANKITITNDKGRLSKEEIERMVQEAERYKAEDEGQREKIAAKNALESYAFNMKSAVGDEGLKDKISESDKNKILDKCNEVLSWLEANQLAEKDEFDHKRKELENMCNPIITKLYQSGCTGPTCTPGYTPGRAATGPTIEEVD

SEQ ID NO: 12 - protein, 641 aa, Rattus Norvegicus
MAANKGMAIGIDLGTTYSCVGVFQHGKVEIIANDQGNRTTPSYVAFTDTERLIGDAAKNQVAMNPQNTVFDAKRLIGRKFNDPVVQSDMKLWPFQVINEAGKPKVLVSYKGEKKAFYPEEISSMVLTKMKETAEAFLGHSVTNAVITVPAYFNDSQRQATKDAGVIAGLNVLRIINEPTAAAIAYGLDKGSHGERHVLIFDLGGGTFDVSILTIDDGIFEVKATAGDTHLGGEDFDNRLVSHFVEEFKRKHKKDISQNKRAVRRLRTACERAKRTLSSSTQANLEIDSLYEGIDFYTSITRARFEELCADLFRGTLEPVEKSLRDAKMDKAKIHDIVLVGGSTRIPKVQKLLQDYFNGRDLNKSINPDEAVAYGAAVQAAILMGDKSEKVQDLLLLDVAPLSLGLETAGGVMTVLIKRNSTIPTKQTQIFTTYSDNQPGVLIQVYEGERAMTRDNNLLGRFDLTGIPPAPRGVPQIEVTFDIDANGILNVTAMDKSTGKANKITITNDKGRLSKEEIERMVQEAERYKAEDEGQREKIAAKNALESYAFNMKSAVGDEGLKDKISESDKKKILDKCSEVLSWLEANQLAEKEEFDHKRKELENMCNPIITKLYQSGCTGPTCAPGYTPGRAATGPTIEEVD

(19) United States
(12) Patent Application Publication
(10) Pub. No.: US 2009/0291442 A1
(43) Pub. Date: Nov. 26, 2009

(54) HSPA1A AS A MARKER FOR SENSITIVITY TO KSP INHIBITORS

(75) Inventors: Priti S. Hegde, San Francisco, CA (US); Jeffrey R. Jackson, Collegeville, PA (US); Jessica R. Schroeck, Collegeville, PA (US)

Correspondence Address: SMITHKLINE BEECHAM CORPORATION;CORPORATE INTELLECTUAL PROPERTY-US, UW2220, P. O. BOX 1539, KING OF PRUSSIA, PA, 19406-0939, US

(73) Assignee: SMITHKLINE BEECHAM CORPORATION, Philadelphia, PA (US)

(21) Appl. No.: 12/375,025

(22) PCT Filed: Jul. 26, 2007
(86) PCT No.: PCT/US07/74418

§ 371 Date: Jan. 26, 2009

Related U.S. Application Data
(60) Provisional application No. 60/820,502, filed on Jul. 27, 2006.

(51) Int. Cl. C12Q 1/68

(52) U.S. Cl. 435/6

(57) ABSTRACT

The present invention relates to methods for predicting a response to treatment with a kinesin spindle protein inhibitor using heat shock protein 70, isoform A1a, also known as HSPA1a, as a marker for sensitivity to the kinesin spindle protein (KSP) inhibitors. Method are provided for predicting a response to treatment with a kinesin spindle protein inhibitor of a first mammal in need thereof comprising determining an amount of HSPA1a mRNA transcript produced by said first mammal, wherein the amount of said HSPA1a mRNA transcript produced by said first mammal is indicative of said mammal's sensitivity to said kinesin spindle protein inhibitor

### FIELD OF INVENTION

The present invention relates to methods for predicting a response to treatment with a kinesin spindle protein inhibitor using heat shock protein 70 kDa, isoform A1a, also known as HSPA1a, as a marker for sensitivity to the kinesin spindle protein (KSP) inhibitors.

### BACKGROUND OF THE INVENTION

With the high prevalence of cancer in the world and the various cancer therapeutics on the market, the need to identify the best treatment for a patient arises. Markers that can predict how well a patient will respond to certain therapeutics will greatly help treat patients more effectively in addition to helping choose patients for clinical trials.

Kinesin spindle protein (KSP) is the mitotic kinesin motor protein involved in centrosome separation, one of the earliest steps in the mitotic process (Blangy, et al., Cell. 1995; 83:1159-1169.). When centrosomes migrate toward opposite poles, a bipolar mitotic spindle is formed. If formation does not occur, then mitosis is arrested. Therefore, inhibitors of kinesin motor proteins such as Kinesin Spindle Protein (KSP) offer an attractive alternative as a new generation of mitotic inhibitors.

Heat shock 7OkDa protein 1A (HSPA1a) is an example of a marker for sensitivity to the kinesin spindle protein (KSP) inhibitors. HSPA1a functions to help stabilize proteins against aggregation and mediate protein folding. Other gene aliases include: HSP72; HSPA1; HSPA1B; and HSP70-1. Although there are eight known isoforms of heat shock protein 70 kDa (HSP70) in the human genome, only human HSPA1a (SEQ ID NO.1) is associated with sensitivity to the KSP inhibitors. Hence there is a need for methods for predicting a patient's sensitivity to KSP inhibitors.

### SUMMARY OF THE INVENTION

In one aspect of the present invention, methods are provided for predicting a response to treatment with a kinesin spindle protein inhibitor of a first mammal in need thereof comprising determining an amount of heat shock 70 kDa, isoform A1a (HSPA1a) mRNA transcript produced by said first mammal, wherein the amount of said HSPA1a mRNA transcript produced by said first mammal is indicative of said mammal's sensitivity to said kinesin spindle protein inhibitor.

In another aspect of the present invention methods are provided for treating a cell proliferative disorder in a first mammal with a kinesin spindle protein inhibitor comprising predicting a clinical response to treatment with said kinesin spindle protein inhibitor comprising determining an amount of HSPA1a mRNA transcript produced by said first mammal, wherein the amount of said HSPA1a mRNA transcript produced by said first mammal is indicative of said mammal's sensitivity to treatment with said kinesin spindle protein inhibitor.

In yet another aspect, the present invention provides a kit for predicting a clinical response of a mammal to treatment of a cellular proliferative disorder with a kinsesin spindle protein inhibitor comprising a reagent capable of detecting an amount of HSPA1a mRNA transcript in a tissue sample from said mammal, wherein the amount of said HSPA1a mRNA transcript in said tissue sample is indicative of said mammal's sensitivity to said kinesin spindle protein inhibitor.

### BRIEF DESCRIPTION OF THE FIGURES

FIG. 1 shows an amino acid sequence of human HSPA1a (SEQ ID NO.1).

FIG. 2 shows the expression levels of human HSPA1a (SEQ ID NO:1) in Wilm's tumor samples.

FIG. 3 shows the expression levels of human HSPA1a (SEQ ID NO:1) in pre-clinical cell lines.

FIG. 4 shows amino acid alignment for mammalian heat shock proteins (SEQ ID NOs:1-12) including HSPA1A, HSPA1B, HSPA1L from human (designated as Hs), African green monkey (designated as ca), mouse (designated as mm), rat (designated as rn) and cow (designated as bt).

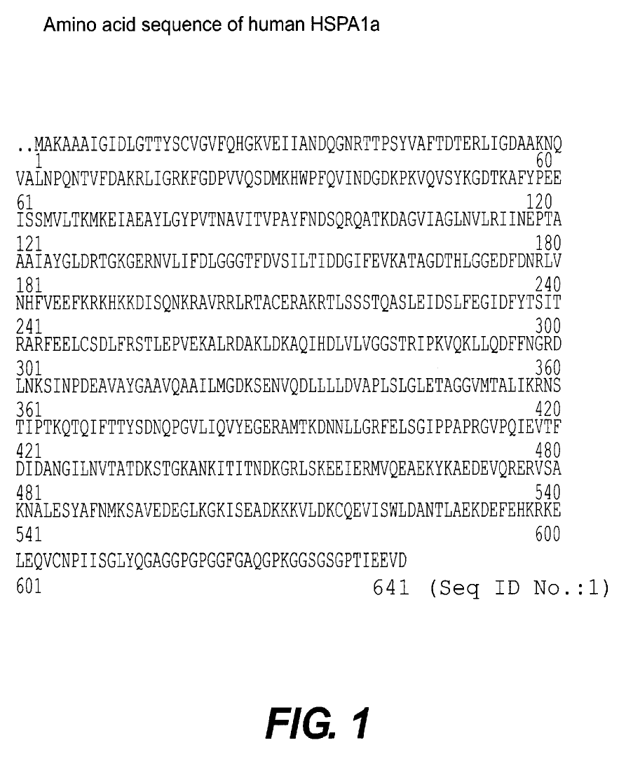

### DESCRIPTION OF THE INVENTION

The present invention provides a variety of methods for predicting a mammal's response to treatment with a kinesin spindle protein inhibitor. Methods are also provided for treating cell proliferative disorders in a mammal. The present invention also provides kits for predicting a clinical response of a mammal to treatment of a cellular proliferative disorder with a kinsesin spindle protein inhibitor.

### Definitions

As used herein and as is understood in the art “statistically significantly lower” refers a likelihood that a certain result occurs due to chance alone is less than five times out of 100 (p<0.05). A statistical p-value, which is a measure of probability that an observed difference between groups occurred by chance alone, can be calculated by a number of statistical algorithms that are understood in the art.

As used herein “methods for gene expression profiling” refers to any method capable of measuring either the amount of gene expression of a particular gene in at least one cell. Such methods include, but are not limited to, RT-PCR and microarrays. Such methods also may include, but are not limited to, qualitatively or quantitatively measuring mRNA expression or polypeptide expression, including precursor or mature polypeptide expression, from a particular gene.

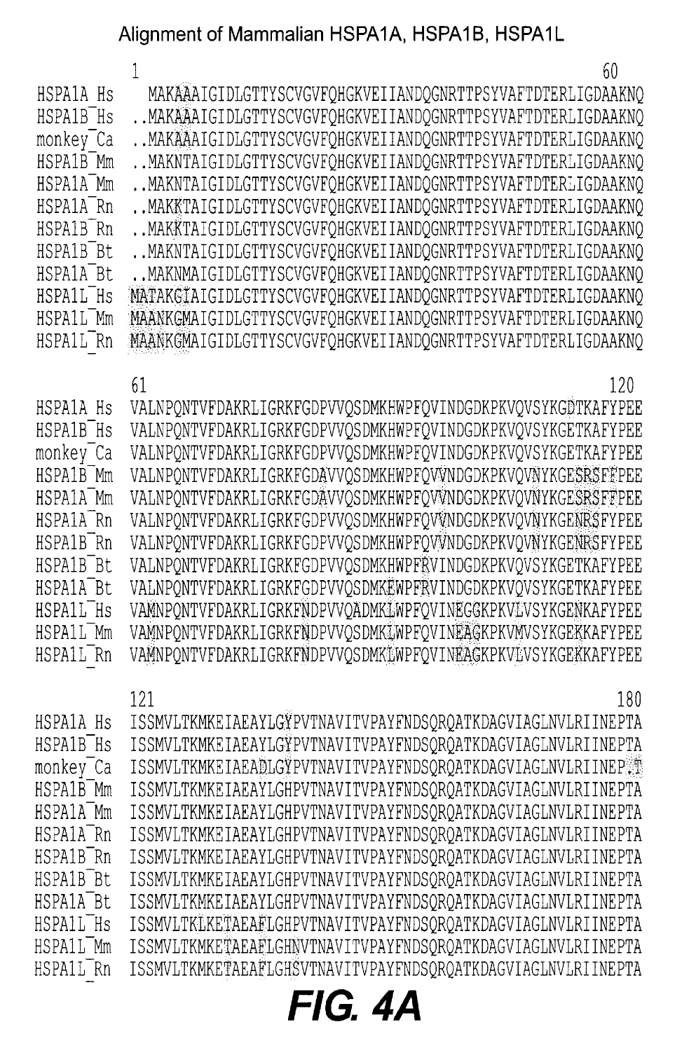

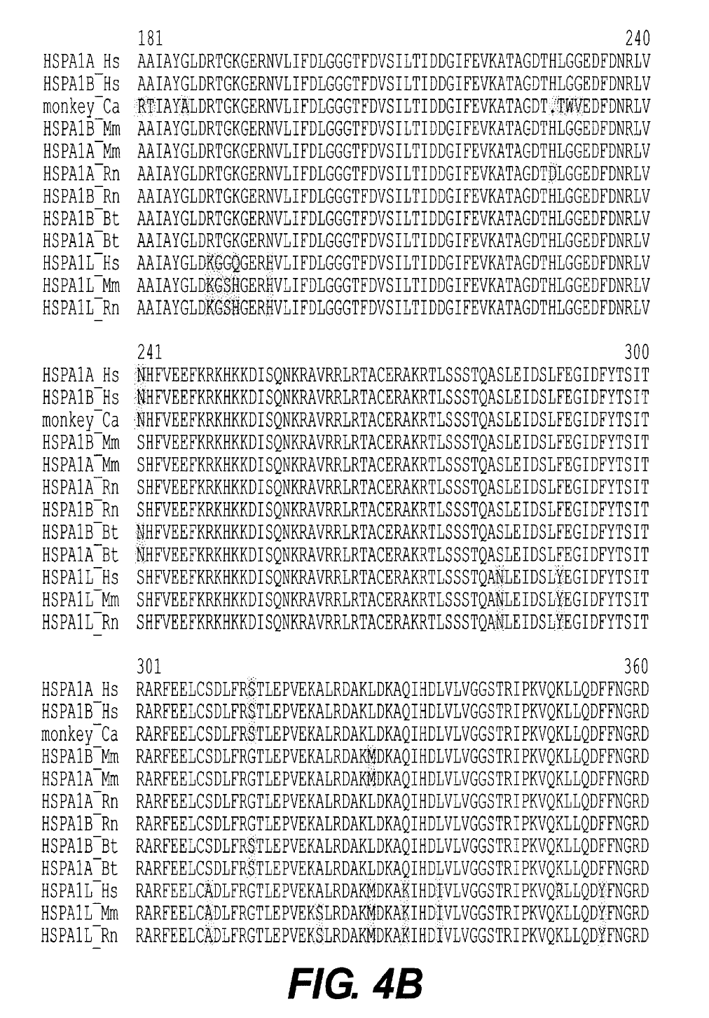

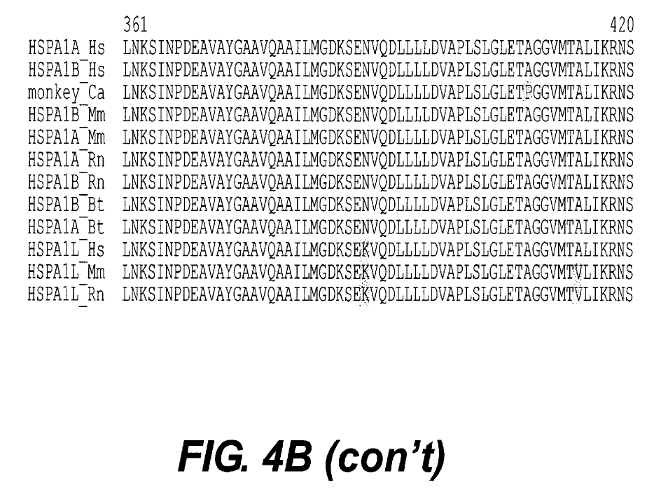

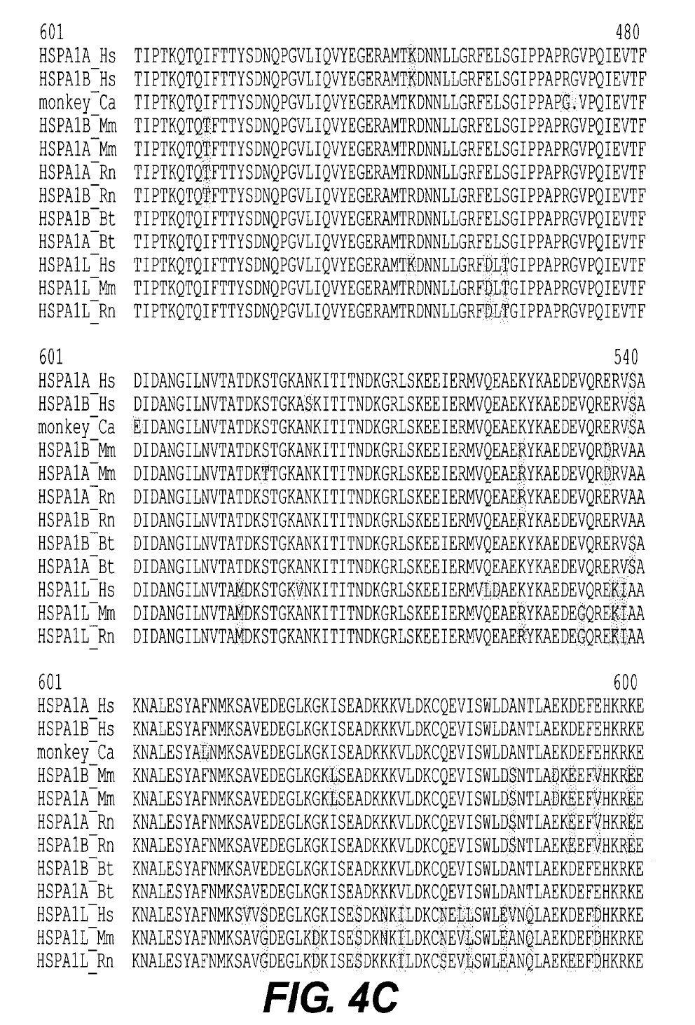

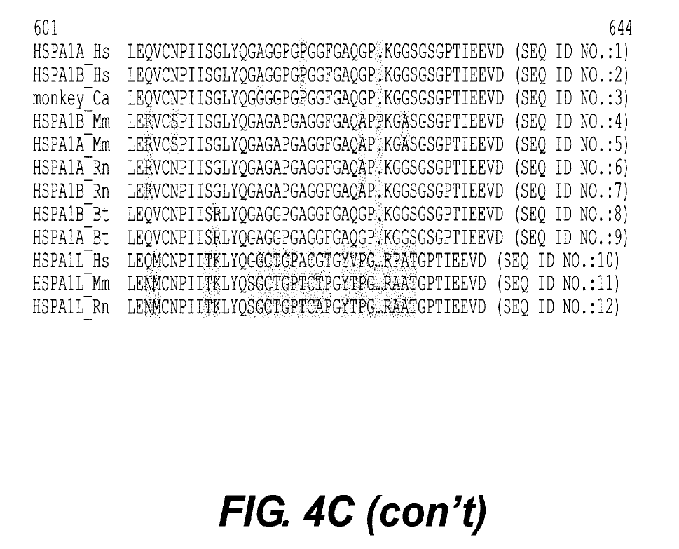

It will be understood by those in the art that the level of a certain protein produced by a cell is related to the level of the messenger RNA (mRNA) which encodes it. Therefore, when the amino acid sequence of the protein such as HSPA1a (SEQ ID NO.:1) is known, methods can easily be envisioned by which production in at least one tumor cell would be determined by measuring levels of the corresponding mRNA for that protein. In addition, complementary DNA for each mRNA relating to a certain protein can also be the specific recognition elements, and the existing techniques known as Northern blots, slot blots, in situ hybridizations, and polymerase chain reactions (PCR) could be applied to determine protein level. Messenger RNA levels have been used to determine production of corresponding proteins (G. Bevilacqua, M. E. Sobel, L. A. Liotta and T. S. Steeg, Cancer Res. 49, 5185-5190 (1989)). Sequence alignment for HSPA1a from human (SEQ ID NO.:1) compared with other mammalian heat shock proteins (SEQ ID NOs:2-12) including HSPA1a, HSPA1b, HSPA1L from human (designated as Hs), African green monkey (designated as ca), mouse (designated as mm), rat (designated as rn) and cow (designated as bt) is presented in FIGS. 4A-C.

As used herein “cell proliferative disorder” refers to excessive proliferation of cells and turnover of cellular matrix, which may contribute to the pathogenesis of several diseases, including cancer, atherosclerosis, rheumatoid arthritis and psoriasis.

The mitotic spindle is a clinically validated anticancer drug target and its disruption is one of the more successful strategies to target tumor cells (Wood, et al., Curr Opin Pharmacol. 2001; 1:370-377). The mitotic spindle is comprised of microtubules, microtubule-associated proteins and motor proteins, including many mitotic kinesins. In order for the cell cycle to progress through mitosis, proper formation of the mitotic spindle is needed (Wood, et al., supra).

Kinesin spindle protein (KSP) is the mitotic kinesin motor protein involved in centrosome separation, one of the earliest steps in the mitotic process (Blangy, et al., Cell. 1995; 83:1159-1169.). When centrosomes migrate toward opposite poles, a bipolar mitotic spindle is formed. If formation does not occur, then mitosis is arrested. Therefore, inhibitors of kinesin motor proteins such as Kinesin Spindle Protein (KSP) offer an attractive alternative as a new generation of mitotic inhibitors.

Tumor biopsies contain mRNA which can be extracted and used to measure the level of expression of genes in that particular tumor. Preliminary analysis of baseline gene expression using xenografts of human Wilm's tumors shows that HSPA1a expression is high in tumors resistant to the KSP inhibitor and absent in tumors sensitive to the inhibitor yielding in a 20-30 fold difference in baseline expression between the different tumors. Further work on preclinical cell lines with known outcomes to the KSP inhibitor confirms the ability of a single gene transcript, HSPA1a, to differentiate responders from non-responder cell lines with ˜91% confidence. Thus, as shown by the present invention HSPA1a, an isoform of HSP70, serves as an effective marker of response to KSP inhibitors.

Thus, the current invention provides methods for predicting a response to treatment with a kinesin spindle protein inhibitor of a first mammal in need thereof comprising determining an amount of HSPA1a mRNA transcript produced by said first mammal, wherein the amount of said HSPA1a mRNA transcript produced by said first mammal is indicative of said mammal's sensitivity to said kinesin spindle protein inhibitor. The HSPA1a mRNA transcript may be produced by at least one tumor cell from said first mammal. The mammal may suffer from a disease selected from the group of: kidney cancer, colon cancer, lung cancer, and breast cancer. The amount of HSPA1a mRNA transcript produced by said at least one tumor cell from said first mammal may be determined by a variety of gene expression profiling techniques, including but not limited to, Affymetrix® or Taqman gene expression profiling. Tumor cells may be selected from the group of, but not limited to: Wilm's tumors, MX1, MV522, OVCAR-3, PC-3, SK-OV-3, MCF7, HT-29, A549, A498, COL0201, COL0205, HL-60, JURKAT, LNCaP, MOLT-4, RAJI, SW-620, THP-1, and U937.

The amount of HSPA1a mRNA transcript produced by said at least one tumor cell from said first mammal may be statistically compared with the an amount of HSPA1a mRNA transcript produced by at least one tumor cell from a second mammal by a variety of statistical methods known in the art. These methods include, but are not limited to, student t-test or an ANOVA comparison. Thus, in one aspect of the present invention, the amount of HSPA1a mRNA transcript produced by said at least one tumor cell from said first mammal is statistically significantly lower than an amount of HSPA1a mRNA transcript produced by at least one tumor cell from a second mammal, wherein said second mammal is resistant to treatment with said kinsesin spindle protein inhibitor. The amount of HSPA1a mRNA transcript produced by said at least one tumor cell from said first mammal compared to the amount of HSPA1a mRNA transcript produced by at least one tumor cell from a second mammal may have a statistical p-value ≦0.05. In one aspect of the present invention, the first mammal and said second mammal are human.

Another aspect of the present invention provides methods for treating a cell proliferative disorder in a first mammal with a kinesin spindle protein inhibitor comprising predicting a clinical response to treatment with said kinesin spindle protein inhibitor comprising determining an amount of HSPA1a mRNA transcript produced by said first mammal, wherein the amount of said HSPA1a mRNA transcript produced by said first mammal is indicative of said mammal's sensitivity to treatment with said kinesin spindle protein inhibitor. The HSPA1a mRNA transcript may be produced by at least one tumor cell from said first mammal. The cellular proliferative disorder may be selected from: kidney cancer, colon cancer, lung cancer, and breast cancer. Tumor cells include, but are not limited to, Wilm's tumor cell, MX1, MV522, OVCAR-3, PC-3, SK-OV-3, MCF7, HT-29, A549, A498, COLO201, COL0205, HL-60, JURKAT, LNCaP, MOLT-4, RAJI, SW-620, THP-1, and U937. In another aspect of the present invention, the first mammal is predicted to be sensitive to treatment with said kinsesin spindle protein inhibitor if the amount of HSPA1a mRNA transcript produced by said at least one tumor cell from said first mammal is statistically significantly lower than an amount of HSPA1a mRNA transcript produced by at least one tumor cell from a second mammal, wherein said second mammal is resistant to treatment with said kinsesin spindle protein inhibitor. The amount of HSPA1a mRNA transcript produced by said at least one tumor cell from said first mammal compared to the amount of HSPA1a mRNA transcript produced by at least one tumor cell from a second mammal may have a p-value ≦0.05. In one aspect of the present invention, the first mammal and said second mammal are human.

In yet another aspect, this invention provides a kit for predicting a clinical response of a mammal to treatment of a cellular proliferative disorder with a kinsesin spindle protein inhibitor comprising a reagent capable of detecting an amount of HSPA1a mRNA transcript in a tissue sample from said mammal, wherein the amount of said HSPA1a mRNA transcript in said tissue sample is indicative of said mammal's sensitivity to said kinesin spindle protein inhibitor. In one aspect, the tissue sample comprises at least one tumor cell. In another aspect, the mammal is human.

The following examples illustrate various aspects of this invention. These examples, while illustrative, do not limit the scope of this invention, which is defined by the appended claims.

### EXAMPLES

### Example 1

Ispinesib mesylate is a potent and selective inhibitor of KSP in clinical development for the treatment of cancer (Johnson, et al. Proc Am Assoc Cancer Res. 2002;43;269. Abstract 1335; Jackson, et al. Proc Am Assoc Cancer Res. 2002;43;269. Abstract 1336; Gonzales, et al. Proc Am Assoc Cancer Res. 2002;43;269. Abstract 1337.). KSP acts to force apart the two centrosomes of the emerging mitotic spindle. Ispinesib inhibition of KSP prevents formation of a bipolar mitotic spindle thus arresting cell cycle (Johnson, et al., supra; Jackson, et al., supra; Gonzales, et al, supra.).

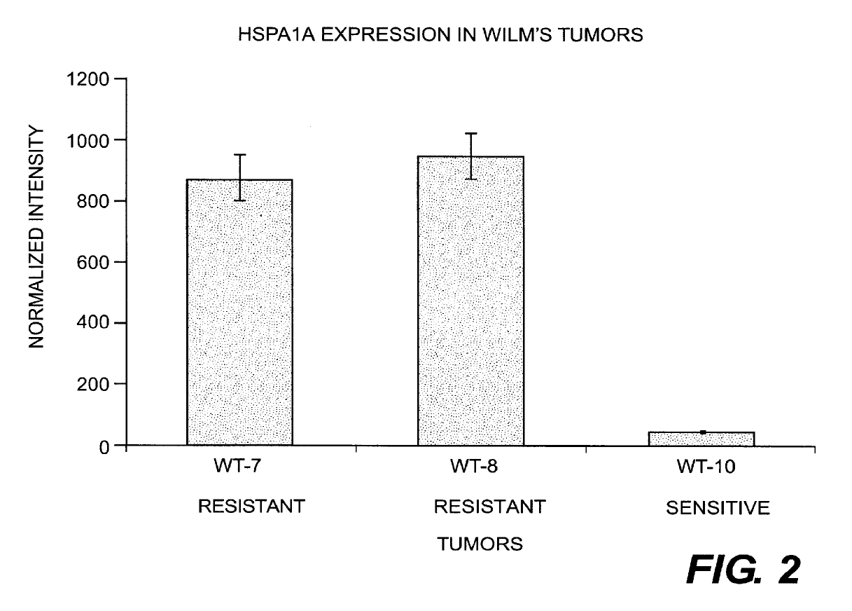

Affymetrix® gene expression profiling was conducted to identify baseline expression patterns associated with sensitivity to the KSP inhibitor in human Wilm's tumor xenograft models. Total RNA from three distinct Wilm's tumors with known outcomes to Ispinesib mesylate (2-3 replicates) inhibition was labeled using a 5 μg protocol and hybridized to the HG-U133A Affymetrix® array. Baseline gene expression differences between tumors resistant (WT7 and WT8) and sensitive (WT10) to Ispinesib mesylate were compared. Sorting the data based on the fold change, HSPA1a had a 29 fold change difference between resistant and sensitive tumors (as shown in FIG. 2). Wilm's tumors sensitive to the compound had an average expression of 914.33, versus an average expression of 30.95 in resistant tumors indicating that low expression is associated with sensitivity. Validation by QPCR of the same tumors confirmed this expression pattern.

### Example 2

Baseline expression of HSPA1a was validated in 4 cell lines: MX1 (breast cancer) and MV522 (lung cancer) resistant to Ispinesib mesylate and Colo201 (colon cancer) and Colo205 (colon cancer) sensitive to Ispinesib mesylate inhibition. Like the tumors, HSPA1a expression differentiated the resistant cell lines from the sensitive cell lines. Cell lines resistant to the KSP inhibitor had an average gene intensity of 930.92 whereas the cell lines that were sensitive had an average gene intensity of 17.2.

### Example 3

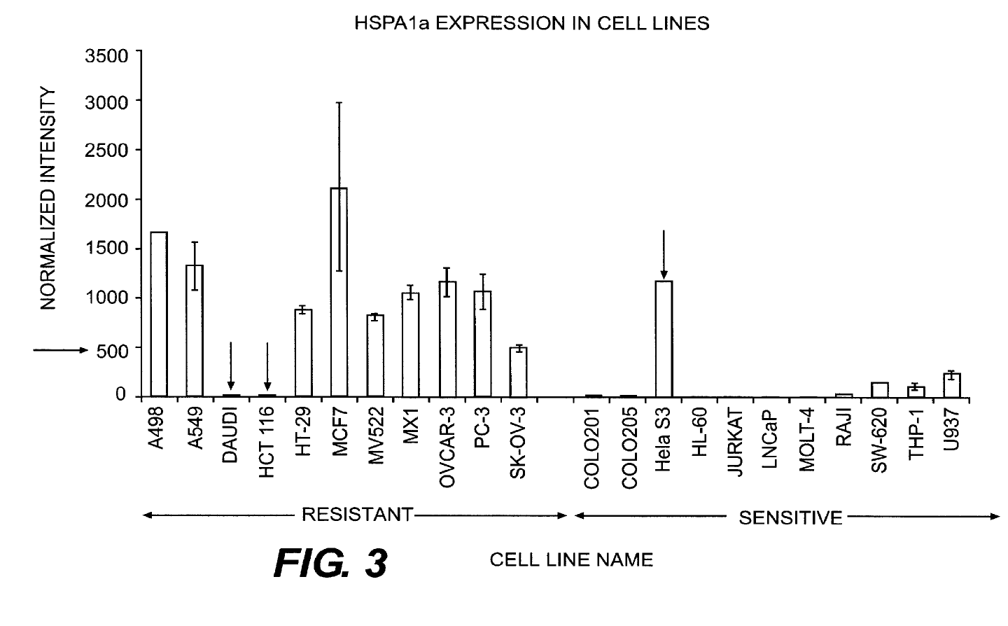

A more extensive list of preclinical cell lines compared with those presented in Example 2, including cell lines from both solid and liquid tumors, was analyzed. Again, these cell lines were previously determined to be either resistant or sensitive to Ispinesib mesylate using FACS analysis. FIG. 3 shows the expression of HSPA1a in these cell lines. Of the 22 cell lines analyzed, HSPA1a expression levels were correctly able to classify 19 cell lines as resistant or sensitive to the KSP inhibitor. The HSPA1a marker misclassified 2 cell lines Daudi and HCT116 as sensitive (due to low baseline expression in these cells) and HeLaS3 cell line as resistant (due to high baseline expression of HSPA1a in these cells).

Any patent application to which this application claims priority is incorporated by reference herein in its entirety.

1. A method for predicting a response to treatment with a kinesin spindle protein inhibitor of a first human in need thereof comprising determining an amount of HSPA1a mRNA transcript produced by said first human, wherein the amount of said HSPA1a mRNA transcript produced by said first human is indicative of said human's sensitivity to said kinesin spindle protein inhibitor.

2. The method of claim 1, wherein said HSPA1a mRNA transcript is produced by at least one tumor cell from said first human.

3. The method of claim 1, wherein said first human suffers from a disease selected from the group of: kidney cancer, colon cancer, lung cancer, and breast cancer.

4. The method of claim 2, wherein the amount of HSPA1a mRNA transcript produced by said at least one tumor cell from said first human is determined by gene expression profiling.

5. The method of claim 4, wherein the method of gene expression profiling is selected from the group of: Affymetrix® and Taqman gene expression profiling.

6. The method of claim 2, wherein said at least one tumor cell from said first human is of the type selected from the group of: Wilm's tumor cell, MX1, MV522, OVCAR-3, PC-3, SK-OV-3, MCF7, HT-29, A549, A498, COLO201, COL0205, HL-60, TURKAT, LNCAP, MOLT-4, RAJI, SW-620, THP-1, and U937.

7. The method of claim 2, wherein said first human is predicted to be sensitive to treatment with said kinsesin spindle protein inhibitor if the amount of HSPA1a mRNA transcript produced by said at least one tumor cell from said first human is statistically significantly lower than an amount of HSPA1a mRNA transcript produced by at least one tumor cell from a second human, wherein said second human is resistant to treatment with said kinsesin spindle protein inhibitor.

8. The method of claim 7, wherein the amount of HSPA1a mRNA transcript produced by said at least one tumor cell from said first human compared to the amount of HSPA1a mRNA transcript produced by at least one tumor cell from a second human has a statistical p-value of ≦0.05.

9. (canceled)

10. A method of treating a cell proliferative disorder in a first human with a kinesin spindle protein inhibitor comprising predicting a clinical response to treatment with said kinesin spindle protein inhibitor comprising determining an amount of HSPA1a mRNA transcript produced by said first human, wherein the amount of said HSPA1a mRNA transcript produced by said first human is indicative of said human's sensitivity to treatment with said kinesin spindle protein inhibitor.

11. The method of claim 10, wherein said HSPA1a mRNA transcript is produced by at least one tumor cell from said first human.

12. The method of claim 10, wherein the cellular proliferative disorder is selected from: kidney cancer, colon cancer, lung cancer, and breast cancer.

13. The method of claim 11, wherein said at least one tumor cell is of the type selected from the group of: Wilm's tumor cell, MX1, MV522, OVCAR-3, PC-3, SK-OV-3, MCF7, HT-29, A549, A498, COLO201, COL0205, HL-60, JURKAT, LNCaP, MOLT-4, RAJI, SW-620, THP-1, and U937.

14. The method of claim 11, wherein said first human is predicted to be sensitive to treatment with said kinsesin spindle protein inhibitor if the amount of HSPA1a mRNA transcript produced by said at least one tumor cell from said first human is statistically significantly lower than an amount of HSPA1a mRNA transcript produced by at least one tumor cell from a second human, wherein said second human is resistant to treatment with said kinsesin spindle protein inhibitor.

15. The method of claim 14, wherein the amount of HSPA1a mRNA transcript produced by said at least one tumor cell from said first human compared to the amount of HSPA1a mRNA transcript produced by at least one tumor cell from a second human has a p-value of ≦0.05.

16. (canceled)

17. A kit for predicting a clinical response of a human to treatment of a cellular proliferative disorder with a kinsesin spindle protein inhibitor comprising a reagent capable of detecting an amount of HSPA1a mRNA transcript in a tissue sample from said human, wherein the amount of said HSPA1a mRNA transcript in said tissue sample is indicative of said human's sensitivity to said kinesin spindle protein inhibitor.

18. The kit of claim 17, wherein the tissue sample comprises at least one tumor cell.

19. (canceled)
